(19) United States
(12) Patent Application Publication
(10) Pub. No.: US 2005/0250788 A1
(43) Pub. Date: Nov. 10, 2005

(54) Aqueous outflow enhancement with vasodilated aqueous cavity

(76) Inventors: Hosheng Tu, Newport Coast, CA (US); David Haffner, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/045,417

(22) Filed: Jan. 27, 2005

Related U.S. Application Data
(60) Provisional application No. 60/540,521, filed on Jan. 30, 2004.

(51) Int. Cl. A61K 31/519

(52) U.S. Cl. 514/252.16; 514/262.1

(57) ABSTRACT

A method for enhancing aqueous outflow and thereby lowering intraocular pressure is disclosed. The method comprises vasodilating aqueous veins by dilating or relaxing the smooth muscle of an aqueous cavity. In one embodiment, the step of dilating or relaxing the smooth muscle of the aqueous cavity is accomplished by slowly releasing loaded smooth muscle relaxing drug at an effective dose over time. In another embodiment, the step of dilating the smooth muscle of the aqueous cavity is accomplished by introducing a smooth muscle drug through an implant.

### RELATED APPLICATIONS

This application claims priority from U.S. Provisional Application No. 60/540,521, entitled “Aqueous Outflow Enhancement with Vasodilated Aqueous Cavity,” filed Jan. 30, 2004, the entirety of which is hereby incorporated by reference.

### BACKGROUND OF THE INVENTION

1. Field of the Invention

This invention relates to reducing intraocular pressure within the animal eye. More particularly, this invention relates to a treatment of glaucoma wherein aqueous humor is permitted to flow out of an anterior chamber of the eye through a surgically implanted pathway. Furthermore, this invention relates to a direct delivery of pharmaceuticals to ocular tissue in the eye.

2. Description of the Related Art

The human eye is a specialized sensory organ capable of light reception and able to receive visual images. The trabecular meshwork serves as a drainage channel and is located in the anterior chamber angle formed between the iris and the cornea. The trabecular meshwork maintains a balanced pressure in the anterior chamber of the eye by draining aqueous humor from the anterior chamber.

About two percent of people in the United States have glaucoma. Glaucoma is a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas.

In glaucomas associated with an elevation in eye pressure (intraocular hypertension), the source of resistance to outflow is mainly in the trabecular meshwork. The tissue of the trabecular meshwork allows the aqueous humor (or “aqueous”) to enter Schlemm's canal, which then empties into aqueous collector channels in the posterior wall of Schlemm's canal and then into aqueous veins, which form the episcleral venous system. Aqueous is a transparent liquid that fills the region between the cornea, at the front of the eye, and the lens. The aqueous is continuously secreted by the ciliary body around the lens, so there is a constant flow of aqueous from the ciliary body to the eye's front chamber. The eye's pressure is determined by a balance between the production of aqueous and its exit through the trabecular meshwork (major route) or uveal scleral outflow (minor route). The trabecular meshwork is located between the outer rim of the iris and the back of the cornea, in the anterior chamber angle. The portion of the trabecular meshwork adjacent to Schlemm's canal (the juxtacanilicular meshwork) causes most of the resistance to aqueous outflow.

Glaucoma is grossly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the angle of the anterior chamber remains open, but the exit of aqueous through the trabecular meshwork is diminished. The exact cause for diminished filtration is unknown for most cases of open-angle glaucoma. Primary open-angle glaucoma is the most common of the glaucomas, and it is often asymptomatic in the early to moderately advanced stage. Patients may suffer substantial, irreversible vision loss prior to diagnosis and treatment. However, there are secondary open-angle glaucomas which may include edema or swelling of the trabecular spaces (e.g., from corticosteroid use), abnormal pigment dispersion, or diseases such as hyperthyroidism that produce vascular congestion.

All current therapies for glaucoma are directed at decreasing intraocular pressure. Medical therapy includes topical ophthalmic drops or oral medications that reduce the production or increase the outflow of aqueous. However, these drug therapies for glaucoma are sometimes associated with significant side effects, such as headache, blurred vision, allergic reactions, death from cardiopulmonary complications, and potential interactions with other drugs. When drug therapy fails, surgical therapy is used. Surgical therapy for open-angle glaucoma consists of laser trabeculoplasty, trabeculectomy, and implantation of aqueous shunts after failure of trabeculectomy or if trabeculectomy is unlikely to succeed. Trabeculectomy is a major surgery that is widely used and is augmented with topically applied anticancer drugs, such as 5-flurouracil or mitomycin-C to decrease scarring and increase the likelihood of surgical success.

Approximately 100,000 trabeculectomies are performed on Medicare-age patients per year in the United States. This number would likely increase if the morbidity associated with trabeculectomy could be decreased. The current morbidity associated with trabeculectomy consists of failure (10-15%); infection (a life long risk of 2-5%); choroidal hemorrhage, a severe internal hemorrhage from low intraocular pressure, resulting in visual loss (1%); cataract formation; and hypotony maculopathy (potentially reversible visual loss from low intraocular pressure).

For these reasons, surgeons have tried for decades to develop a workable surgery for the trabecular meshwork.

The surgical techniques that have been tried and practiced are goniotomy/trabeculotomy and other mechanical disruptions of the trabecular meshwork, such as trabeculopuncture, goniophotoablation, laser trabecular ablation, and goniocurretage. These are all major operations and are briefly described below.

Goniotomy and trabeculotomy are simple and directed techniques of microsurgical dissection with mechanical disruption of the trabecular meshwork. These initially had early favorable responses in the treatment of open-angle glaucoma. However, long-term review of surgical results showed only limited success in adults. In retrospect, these procedures probably failed due to cellular repair and fibrosis mechanisms and a process of “filling in.” Filling in is a detrimental effect of collapsing and closing in of the created opening in the trabecular meshwork. Once the created openings close, the pressure builds back up and the surgery fails.

Regarding Trabeculopuncture, Q-switched Neodymiun (Nd) YAG lasers also have been investigated as an optically invasive technique for creating full-thickness holes in trabecular meshwork. However, the relatively small hole created by this trabeculopuncture technique exhibits a filling in effect and fails.

Goniophotoablation and Laser Trabecular Ablation involve the use of an excimer laser to treat glaucoma by ablating the trabecular meshwork. This was demonstrated not to succeed by clinical trial. Hill et al. used an Erbium:YAG laser to create full-thickness holes through trabecular meshwork (Hill et al., Lasers in Surgery and Medicine 11:341-346, 1991). This technique was investigated in a primate model and a limited human clinical trial at the University of California, Irvine. Although morbidity was zero in both trials, success rates did not warrant further human trials. Failure was again from filling in of surgically created defects in the trabecular meshwork by repair mechanisms. Neither of these is a viable surgical technique for the treatment of glaucoma.

Goniocurretage is an ab interno (from the inside), mechanically disruptive technique that uses an instrument similar to a cyclodialysis spatula with a microcurrette at the tip. Initial results were similar to trabeculotomy: it failed due to repair mechanisms and a process of filling in.

Although trabeculectomy is the most commonly performed filtering surgery, viscocanalostomy (VC) and non-penetrating trabeculectomy (NPT) are two new variations of filtering surgery. These are ab externo (from the outside), major ocular procedures in which Schlemm's canal is surgically exposed by making a large and very deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT involve the formation of an opening or hole under the conjunctiva and scleral flap into the anterior chamber, such that aqueous is drained onto the surface of the eye or into the tissues located within the lateral wall of the eye. These surgical operations are major procedures with significant ocular morbidity. When trabeculectomy, VC, and NPT are thought to have a low chance for success, a number of implantable drainage devices have been used to ensure that the desired filtration and outflow of aqueous through the surgical opening will continue. The risk of placing a glaucoma drainage device also includes hemorrhage, infection, and diplopia (double vision).

### SUMMARY OF THE INVENTION

All of the above embodiments and variations thereof have numerous disadvantages and moderate success rates. They involve substantial trauma to the eye and require great surgical skill in creating a hole through the full thickness of the sclera into the subconjunctival space. The procedures are generally performed in an operating room and have a prolonged recovery time for vision.

The complications of existing filtration surgery have inspired ophthalmic surgeons to find other approaches to lowering intraocular pressure.

The trabecular meshwork and juxtacanilicular tissue together provide the majority of resistance to the outflow of aqueous and, as such, are logical targets for surgical removal in the treatment of open-angle glaucoma. In addition, minimal amounts of tissue are altered and existing physiologic outflow pathways are utilized.

As reported in Arch. Ophthalm. (2000) 118:412, glaucoma remains a leading cause of blindness, and filtration surgery remains an effective, important option in controlling the disease. However, modifying existing filtering surgery techniques in any profound way to increase their effectiveness appears to have reached a dead end. The article further states that the time has come to search for new surgical approaches that may provide better and safer care for patients with glaucoma.

What is needed, therefore, is an extended, site specific treatment method for glaucoma that is faster, safer, and less expensive than currently available modalities. It is one object of the invention to provide a drug-eluting trabecular stent with vasodilating capability enabling enhanced aqueous outflow and lowered intraocular pressure.

A device and method are provided for improved treatment of intraocular pressure due to glaucoma. A trabecular stenting device is adapted for implantation within a trabecular meshwork of an eye such that aqueous humor flows controllably from an anterior chamber of the eye to Schlemm's canal, bypassing the trabecular meshwork. In one embodiment, the trabecular stenting device comprises a quantity of pharmaceuticals effective in treating glaucoma, which are controllably released from the device into tissue of Schlemm's canal and/or downstream collector channels. Depending upon the specific treatment contemplated, pharmaceuticals may be utilized in conjunction with the trabecular stenting device such that aqueous flow either increases or decreases as desired. Placement of the trabecular stenting device within the eye and incorporation, and eventual release, of a proven pharmaceutical glaucoma therapy will reduce, inhibit or slow the effects of glaucoma.

One embodiment provides a trabecular stenting device that is implantable within an eye. The device comprises an inlet section containing at least one lumen, an outlet section having at least one outlet end. Optionally, there comprises a flow-restricting member within the lumen that is configured to prevent at least one component of blood from passing through the flow-restricting member. The device may also comprise an intraocular pump to actively pump the aqueous or vasodilation-enhancing agent into aqueous cavity. The device may further comprise a middle section having at least one lumen. The middle section is fixedly attached to the outlet section and the lumen is in fluid communication with the lumen of the outlet section. The middle section is fixedly attached to the inlet section and the lumen within the middle section in fluid communication with the lumen of the inlet section. The device is configured to permit fluid entering the lumen of the inlet section to enter the lumen of the middle section, pass into the lumen of the outlet section, and then exit the outlet section through the at least one outlet end.

A method of treating glaucoma is also provided. In accordance with one method disclosed herein, the method comprises providing at least one pharmaceutical substance incorporated into a trabecular stenting device, implanting the trabecular stenting device within a trabecular meshwork of an eye such that a first end of the trabecular stent is positioned in an anterior chamber of the eye while a second end is positioned in a Schlemm's canal, and allowing the stenting device to release a quantity of the pharmaceutical substance into the eye, particularly into the aqueous cavity. The outlet end of the trabecular stenting device preferably establishes a fluid communication between the anterior chamber and the aqueous cavity.

One method of lowering intraocular pressure disclosed herein comprises positioning an end of a body in an aqueous cavity of an eye and introducing a dilating agent from the body into the aqueous cavity of the eye. The dilating agent may be selected from the group consisting of a phosphodiesterase inhibitor, an alpha-adrenergic antagonist, a serotonin reuptake inhibitor (or an “SSRI”), and an angiotensin converting enzyme (or “ACE”) inhibitor.

A method is also disclosed for regulating aqueous humor outflow within an eye is provided. The method comprises creating an incision in a trabecular meshwork of the eye, wherein the incision is substantially parallel with a circumference of a limbus of the eye, inserting an outlet section of a trabecular stenting device through the incision into Schlemm's canal such that the outlet section resides within Schlemm's canal while an inlet section of the trabecular stenting device resides in the anterior chamber, and initiating an outflow of aqueous humor from the anterior chamber through the trabecular stenting device into Schlemm's canal.

Another method of regulating intraocular pressure within an eye comprises making an incision passing into a trabecular meshwork of the eye, wherein the incision is oriented lengthwise substantially parallel with a circumference of a limbus. The incision establishes a fluid communication between an anterior chamber and Schlemm's canal of the eye. The method further comprises implanting a trabecular stenting device through the incision such that an outlet section of the trabecular stenting device resides within Schlemm's canal and an inlet section of the trabecular stenting device resides within the anterior chamber. The method still further comprises establishing a fluid transfer from the anterior chamber through the trabecular stenting device into Schlemm's canal.

Another aspect provides an apparatus for implanting a trabecular stenting device within an eye. The apparatus comprises a syringe portion and a cannula portion that has proximal and distal ends. The proximal end of the cannula portion is attached to the syringe portion. The cannula portion further comprises a first lumen and at least one irrigating hole disposed between the proximal and distal ends of the cannula portion. The irrigating hole is in fluid communication with the lumen. The apparatus further includes a holder comprising a second lumen for holding the trabecular stenting device. A distal end of the second lumen opens to the distal end of the cannula portion, and a proximal end of the second lumen is separated from the first lumen of the cannula portion. The holder holds the trabecular stenting device during implantation of the device within the eye, and the holder releases the trabecular stenting device when a practitioner activates deployment of the device.

In accordance with another method disclosed herein, the method comprises providing fluid through the lumen of the microstent to therapeutically dilate the aqueous cavity. The term “aqueous cavity” herein refers to any one or more of the downstream aqueous passageways “behind,” or distal, the trabecular meshwork, including, without limitation, Schlemm's canal, aqueous collector channels, and episcleral veins. In one embodiment, the fluid contains therapeutic substance, including pharmaceuticals, genes, growth factors, enzymes and like. In another embodiment, the fluid contains sterile saline, viscoelastic, or the like. The mode of fluid injection may be a pulsed mode, an intermittent mode or a programmed mode. In yet another embodiment, the pressure of the fluid therapy is effective to cause therapeutic effects on the tissue of the aqueous cavity. In one embodiment, the fluid pressure is effective to cause the dilation of the aqueous cavity beyond the tissue elastic yield point for permanent (i.e., plastic) deformation. In other embodiment, the fluid is at an elevated pressure effective to cause plastic deformation for at least a portion of the aqueous cavity. In some embodiments, fluid is infused through a lumen of the stent, and in some embodiments, fluid is time-released from the stent.

In some arrangements the infusing further comprises coupling the inflow portion of the stent with a fluid delivery element that transmits the fluid to the stent. In one embodiment, the coupling comprises securing a screw thread arrangement of the fluid delivery element with a receiving thread arrangement of the stent. In another embodiment, the coupling comprises securing the fluid delivery element snugly with the proximal end of the stent by either inserting one end of the fluid delivery element inside the inner wall or extending one end of the fluid delivery element over the outer wall of the proximal end of the stent.

In certain preferred arrangements, the fluid comprises a therapeutic substance such as a pharmaceutical, a gene, a growth factor, and/or an enzyme. In other preferred arrangements, the fluid comprises a therapeutic substance such as an antiglaucoma drug, a beta-adrenergic antagonist, a TGF-beta compound, an antibiotic, and/or a vasodilation-enhancing agent.

Some embodiments provide that a temperature of the fluid is raised sufficiently to enhance plastic deformation. And some embodiments provide that a pH of the fluid is adjusted sufficiently to enhance the plastic deformation. Yet other embodiments further include vibrating a tissue of the eye.

One aspect includes a method of treating glaucoma, including inserting a stent through an incision in an eye. The stent preferably has an inflow portion that is in fluid communication with an outflow portion of the stent. The method further comprises positioning the stent such that the inflow portion of the stent is positioned in the anterior chamber of the eye and the outflow portion of the stent is positioned at an aqueous cavity and infusing fluid from the inflow portion to the outflow portion of the stent.

In some arrangements the aqueous cavity is Schlemm's canal. In certain arrangements, the method further comprises positioning the stent such that the outflow portion of the stent is in Schlemm's canal. In some arrangements the aqueous cavity is an aqueous collector channel.

Another aspect provides a method of implanting a trabecular stenting device within an eye. The method comprises creating a first incision in a cornea on a first side of the eye, wherein the first incision passes through the cornea into an anterior chamber of the eye. The method further comprises passing an incising device through the first incision and moving a distal end of the incising device across the anterior chamber to a trabecular meshwork residing on a second side of the eye, and using the incising device to create a second incision. The second incision is in the trabecular meshwork, passing from the anterior chamber through the trabecular meshwork into Schlemm's canal. The method further comprises inserting the trabecular stenting device into a distal space of a delivery applicator. The delivery applicator comprises a cannula portion having a distal end and a proximal end attached to a syringe portion. The cannula portion has at least one lumen and at least one irrigating hole disposed between proximal and distal ends of the cannula portion. The irrigating hole is in fluid communication with the lumen. The distal space comprises a holder that holds the trabecular stenting device during delivery and releases the trabecular stenting device when a practitioner activates deployment of the device. The method further comprises advancing the cannula portion and the trabecular stenting device through the first incision, across the anterior chamber and into the second incision, wherein an outlet section of the trabecular stenting device is implanted into Schlemm's canal while an inlet section of the trabecular stenting device remains in fluid communication with the anterior chamber. The method still further comprises releasing the trabecular stenting device from the holder of the delivery applicator.

Some aspects of the invention relate to a method for reducing intraocular pressure by dilating or relaxing the smooth muscle of an aqueous cavity. In one embodiment, the step of dilating or relaxing the smooth muscle of the aqueous cavity is accomplished by slowly releasing (or time-releasing) loaded smooth muscle relaxing drug at an effective dose over time. In another embodiment, the step of dilating or relaxing the smooth muscle of the aqueous cavity is accomplished by injecting fluid through the implanted pathway of a stent or an opening on trabecular meshwork, wherein the fluid contains smooth muscle relaxing drug (a type of vasodilation-enhancing agents) at an effective dose.

Some aspects relate to a method of releasing smooth muscle relaxing drug to the aqueous cavity at an effective dose over time, ab internally, ab externally or through retrograde infusion. Some methods involve vasodilating a tissue of an aqueous cavity comprising administering a vasodilating agent to the aqueous cavity.

Some aspects relate to a method of vasodilating a tissue of an aqueous cavity comprising administering a vasodilating agent to the aqueous cavity, wherein the vasodilating agent is a phosphodiesterase type 5 inhibitor. Some agents that may be used are sertraline, sildenafil, vardenafil, or tadalafil.

Some aspects relate to a method of vasodilating a tissue of an aqueous cavity comprising administering a vasodilating agent to the aqueous cavity, wherein the vasodilating agent is a phosphodiesterase type 5 inhibitor, and wherein the phosphodiesterase type 5 inhibitor is selected from a group consisting of 5-(2-ethoxy-5-morpholinoacetylphenyl)-1-methyl-3-n-propyl-1,6-dihydro-7-H-pyrazolo[4,3-d]pyrimidin-7-one; 3-ethyl-5-[5-(4-ethylpiperazin-1-ylsulphonyl)-2-n-propoxyphenyl]-2-(pyridin-2-yl)methyl-2,6-dihydro-7H-pyrazolo[4,-3-d]pyrimidin-7-one; 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one; (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazol-o[4,3-d]pyrimidin-7-one, also known as 3-ethyl-5-{5-[4-ethylpiperazin-1-yl-sulphonyl]-2-([(1R)-2-methoxy-1-methylethyl]oxy)pyridin-3-yl}-2-methyl-2,-6-dihydro-7H-pyrazolo[4,3-d]pyrimidin-7-one; 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-d-ihydro-2-(2-methoxyethyl)-7-oxo-2H-pyrazolo[4,3-d]pyrimidin-5-yl]-3-pyridy-1-sulphonyl}-4-ethylpiperazine; 5-[2-iso-Butoxy-5-(4-ethylpiperazin-1-yl-sulphonyl)pyridin-3-yl]-3-ethyl-2-(1-methylpiperidin-4-yl)-2,6-dihydro-7H-p-yrazolo[4,3-d]pyrimidin-7-one; 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one; 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one; 5-(5-Acetyl-2-butoxy-3-pyridinyl)-3-ethyl-2-(1-ethyl-3-azetidinyl)-2,6-di-hydro-7H-pyrazolo[4,3-d]pyrimidin-7-one.

Some aspects relate to a method of vasodilating a tissue of an aqueous cavity comprising administering a vasodilating agent to the aqueous cavity, wherein the vasodilating agent is coated on a surface of a trabecular stent having a distal end and a proximal end, wherein the distal end of the trabecular stent is placed in an aqueous cavity and the proximal end of the trabecular stent is placed in an anterior chamber.

Some aspects relate to a method of vasodilating a tissue of an aqueous cavity comprising administering a vasodilating agent to the aqueous cavity, wherein the vasodilating agent is administered topically on an eye configured for diffusing to the aqueous cavity.

Some aspects relate to a method of vasodilating a tissue of an aqueous cavity comprising administering a vasodilating agent to the aqueous cavity, further comprising an anti-glaucoma agent. Some methods further comprising an anti-glaucoma agent, wherein the anti-glaucoma agent comprises beta-blockers selected from a group consisting of betaxolol, S-betaxolol, levobunolol, carteolol, timolol, and combination thereof, prostaglandins selected from a group consisting of metabolite derivatives of arachindonic acid, miotics selected from a group consisting of pilocarpine, carbachol, acetylcholinesterase inhibitors, and combination thereof, sympathomimetics selected from a group consisting of epinephrine, dipivalylepinephxine, and combination thereof, carbonic anhydrase inhibitors selected from a group consisting of acetazolamide, methazolamide, ethoxzolamide, and combination thereof, and/or alpha and alpha/beta adrenergic receptor agonists selected from a group consisting of epinephrine, dipivalylepinephrine, para-amino clonidine, brimonidine and combination thereof.

Some aspects of the invention relate to a method of vasodilating a tissue of an aqueous cavity comprising administering a vasodilating agent to the aqueous cavity, further comprising a non-steroidal or steroidal anti-inflammatory agent selected from a group consisting of suprofen, ketorolac, dexamethasone, rimexolone, tetrahydrocortisol, and combination thereof. Some methods comprise administering an anti-infective agent, such as ciprofloxacin, for example. Further methods comprise administering a vasodilating agent to the aqueous cavity which further comprises a growth factor.

Some aspects of the invention relate to a method of vasodilating a tissue of an aqueous cavity comprising administering a vasodilating agent to the aqueous cavity, further comprising an anti-allergic agent selected from a group consisting of cromolyn sodium, emedastine, olopatadine, and combination thereof.

Further features, advantages, and embodiments of the present invention will become apparent to one of skill in the art in view of the Detailed Description that follows, when considered together with the attached drawings and claims.

### BRIEF DESCRIPTION OF THE DRAWINGS

FIG. 1 is a coronal, cross-sectional view of an eye.

FIG. 2 is an enlarged cross-sectional view of an anterior chamber angle of the eye of FIG. 1.

FIG. 3 is an oblique elevation view of one embodiment of a trabecular stenting device.

FIG. 4 is an oblique elevation view of another embodiment of a trabecular stenting device.

FIG. 5A is an oblique elevation view of placement of one end of a trabecular stenting device through a trabecular meshwork.

FIG. 5B is an oblique elevation view of placement of one end of a trabecular stenting device through a trabecular meshwork, wherein the trabecular stenting device is passed over a guidewire.

FIG. 6 is an oblique elevation view of a preferred implantation of a trabecular stenting device through a trabecular meshwork.

FIG. 7 is an enlarged, cross-sectional view of a preferred method of implanting a trabecular stenting device within an eye.

FIG. 8 is a perspective view of an anterior chamber angle of an eye, illustrating a trabecular stenting device positioned within a trabecular meshwork.

FIG. 9 is a close-up, cut-away view of an inlet section of the trabecular stenting device of FIGS. 3 and 4, illustrating a flow-restricting member retained within a lumen of the trabecular stenting device.

FIG. 10 shows effects of constriction of an outlet of Schlemm's canal.

FIG. 11 is an oblique elevation view of one embodiment of an axisymmetric trabecular microstent.

FIG. 12 is a detailed view of the proximal section of the microstent of FIG. 11.

FIG. 13 is an applicator for delivering a microstent and infusing fluid for therapeutic treatment.

FIG. 14 is an enlarged, cross-sectional view of a preferred method of implanting a trabecular microstent within an eye.

### DETAILED DESCRIPTION OF THE PREFERRED EMBODIMENTS

The preferred embodiments of the present invention described below relate particularly to surgical and therapeutic treatment of glaucoma through reduction of intraocular pressure. While the description sets forth various embodiment specific details, it will be appreciated that the description is illustrative only and should not to be construed in any way as limiting the invention. Furthermore, various applications of the invention, and modifications thereto, which may occur to those who are skilled in the art, are also encompassed by the general concepts described below.

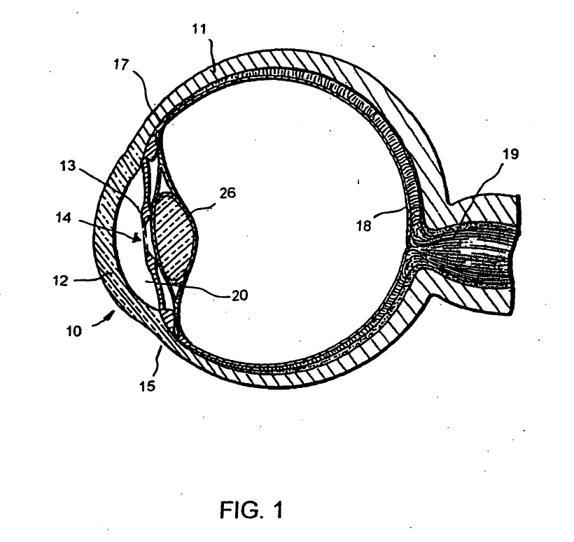

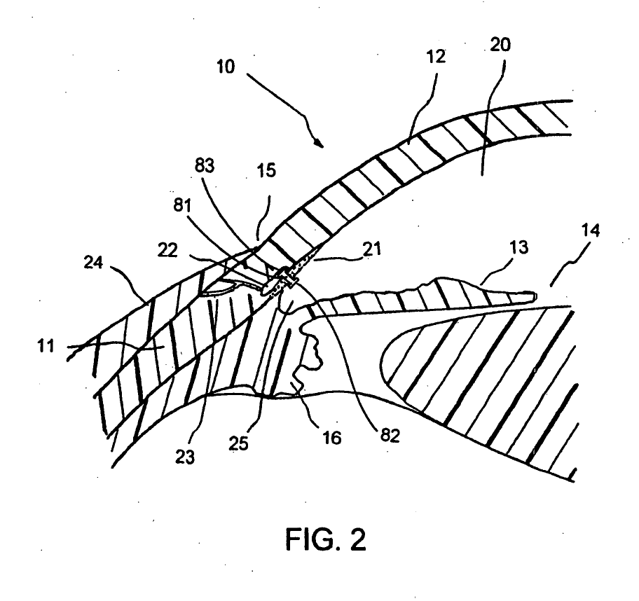

FIG. 1 is a cross-sectional view of an eye 10, while FIG. 2 is a close-up view showing the relative anatomical locations of a trabecular meshwork 21, an anterior chamber 20, and a Schlemm's canal 22. A sclera 11 is a thick collagenous tissue which covers the entire eye 10 except a portion which is covered by a cornea 12. The cornea 12 is a thin transparent tissue that focuses and transmits light into the eye and through a pupil 14, which is a circular hole in the center of an iris 13 (colored portion of the eye). The cornea 12 merges into the sclera 11 at a juncture referred to as a limbus 15. A ciliary body 16 extends along the interior of the sclera 11 and is coextensive with a choroid 17. The choroid 17 is a vascular layer of the eye 10, located between the sclera 11 and a retina 18. An optic nerve 19 transmits visual information to the brain and is the anatomic structure that is progressively destroyed by glaucoma.

The anterior chamber 20 of the eye 10, which is bound anteriorly by the cornea 12 and posteriorly by the iris 13 and a lens 26, is filled with aqueous humor (or “aqueous”). Aqueous is produced primarily by the ciliary body 16, then moves anteriorly through the pupil 14 and reaches an anterior chamber angle 25, formed between the iris 13 and the cornea 12. In a normal eye, aqueous is removed from the anterior chamber 20 through the trabecular meshwork 21. Aqueous passes through the trabecular meshwork 21 into Schlemm's canal 22 and thereafter through a plurality of aqueous veins 23, which merge with blood-carrying veins, and into systemic venous circulation. Intraocular pressure is maintained by an intricate balance between secretion and outflow of aqueous in the manner described above. Glaucoma is, in most cases, characterized by an excessive buildup of aqueous in the anterior chamber 20 which leads to an increase in intraocular pressure. Fluids are relatively incompressible, and thus intraocular pressure is distributed relatively uniformly throughout the eye 10.

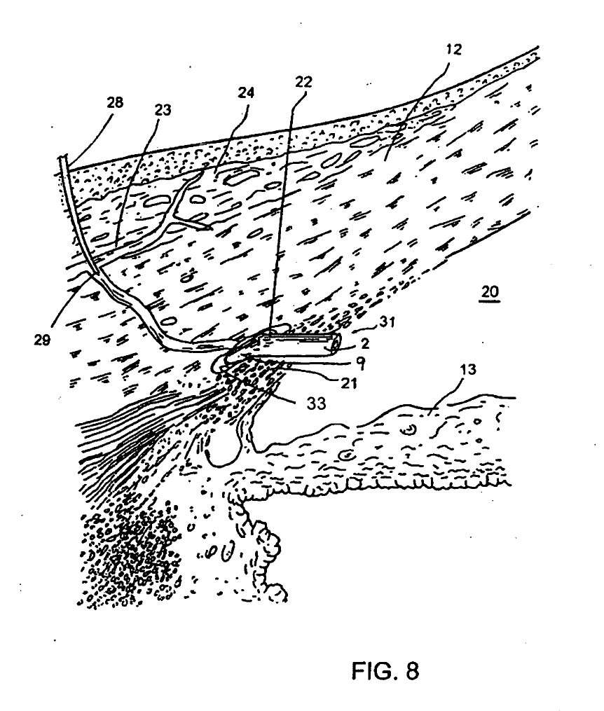

As shown in FIG. 2, the trabecular meshwork 21 is adjacent a small portion of the sclera 11. Exterior to the sclera 11 is a conjunctiva 24. Traditional procedures that create a hole or opening for implanting a device through the tissues of the conjunctiva 24 and sclera 11 involve extensive surgery, as compared to surgery for implanting a device, as described herein, which ultimately resides entirely within the confines of the sclera 11 and cornea 12. FIG. 2 generally illustrates the use of one embodiment of a trabecular stenting device 81 for establishing an outflow pathway, passing through the trabecular meshwork 21, which is discussed in greater detail below. Furthermore, FIG. 8 shows another embodiment of a trabecular stent 31 and a method for retrogradely infusing a pharmaceutical drug (for example, a vasodilating agent) into the aqueous cavity through an infusing apparatus 28, wherein the distal opening 29 of the infusing apparatus 28 is suitably placed at an aqueous vein 23 of the aqueous cavity. Some aspects of the invention relate to a method of vasodilating a tissue of an aqueous cavity comprising administering a vasodilating agent to the aqueous cavity, wherein the vasodilating agent is administered retrogradely via an aqueous vein.

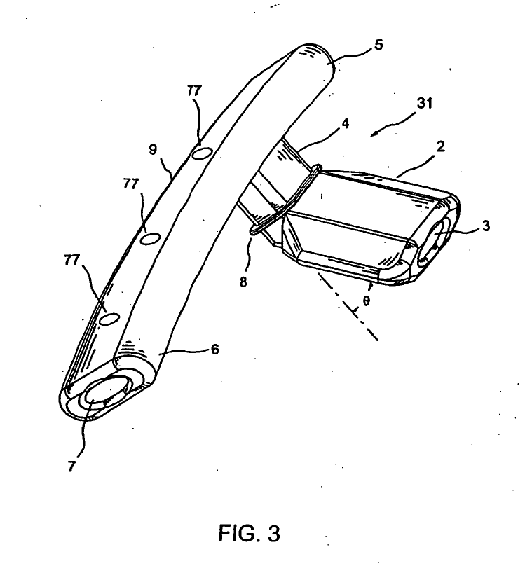

FIG. 3 illustrates a preferred embodiment of a trabecular stenting device 31 which facilitates the outflow of aqueous from the anterior chamber 20 into Schlemm's canal 22, and subsequently into the aqueous collectors and the aqueous veins so that intraocular pressure is reduced. In the illustrated embodiment, the trabecular stenting device 31 comprises an inlet section 2, having an inlet opening 3, a middle section 4, and an outlet section 9. The middle section 4 may be an extension of, or may be coextensive with, the inlet section 2. The outlet section 9 is preferably somewhat flexible to facilitate positioning of the outlet section 9 within an outflow pathway of the eye 10. The outlet section 9 is preferably substantially perpendicular to the middle section 4. “Substantially perpendicular,” as used herein, is defined as subtending an angle between longitudinal axes of the sections 4, 9 ranging between about 30 degrees and about 150 degrees. The device 31 further comprises at least one lumen 7 within sections 4 and 9 which is in fluid communication with the inlet opening 3 of section 2, thereby facilitating transfer of aqueous through the device 31.

The outlet section 9 preferably has a first outlet end 6 and a second, opposite outlet end 5. The lumen 7 within the outlet section 9 opens to at least one of the outlet ends 5, 6. Furthermore, the outlet section 9 may have a plurality of side openings 77, each of which is in fluid communication with the lumen 7, for transmission of aqueous. The middle section 4 is connected to or coextensive with the outlet section 9 and is disposed between the first outlet end 6 and the second outlet end 5. In a preferred embodiment, the outlet section 9 is curved around a point, or curve center, and the middle section 4 extends substantially along a plane that contains the curve center. In this embodiment, the outlet section 9 has a radius of curvature ranging between about 4 mm and about 10 mm.

As will be apparent to a person skilled in the art, the lumen 7 and the remaining body of the outlet section 9 may have a cross-sectional shape that is oval, circular, or other appropriate shape. The cross-sectional shapes of the lumen 7 and the outlet section 9 preferably conform to the shape of the outflow pathway into which the outlet section 9 is placed. The opening of the lumen 7 of the outlet ends 5, 6 may be ovoid in shape to match the contour of Schlemm's canal 22. Further, an outer contour of the outlet section 9 may be elliptical (e.g., ovoid) in shape to match the contour of Schlemm's canal 22. This serves to minimize rotational movement of the outlet section 9 within Schlemm's canal 22, and thereby stabilizes the inlet section 2 with respect to the iris and cornea.

A circumferential ridge 8 is provided at the junction of the inlet section 2 and the middle section 4 to facilitate stabilization of the device 31 once implanted within the eye 10. Preferably, the middle section 4 has a length (between the ridge 8 and the outlet section 9) that is roughly equal to a thickness of the trabecular meshwork 21, which typically ranges between about 100 μm and about 300 μm, yet the length may be less than about 100 μm and greater than about 300 μm. In addition, the outlet section 9 may advantageously be formed with a protuberance or spur projecting therefrom so as to further stabilize the device 31 within the eye 10 without undue suturing.

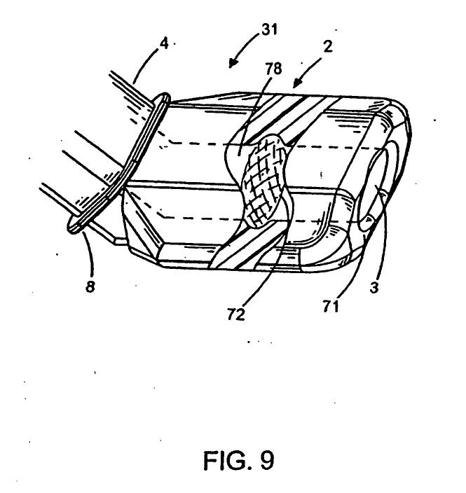

FIG. 9 is a close-up view of the inlet section 2 of the trabecular stenting device 31, illustrating a flow-restricting member 72 which is tightly retained within a lumen 78. The flow-restricting member 72 is shown located close to an inlet side 71 of the inlet section 2. The flow-restricting member 72 serves to selectively restrict at least one component in blood from moving retrograde, i.e., from the outlet section 9 into the anterior chamber 20 of the eye 10. Alternatively, the flow-restricting member 72 may be situated in any location within the device 31 such that blood flow is restricted from retrograde motion. The flow-restricting member 72 may, in other embodiments, be a filter made of a material selected from the following filter materials: expanded polytetrafluoroethylene, cellulose, ceramic, glass, Nylon, plastic, and fluorinated material such as polyvinylidene fluoride (“PVDF”) (e.g., KYNAR™, by DuPont). In another embodiment, the passive flow-restricting member of the trabecular stenting device 31 may be substituted with an active intraocular pump to manage and control the intraocular pressure at a desired level.

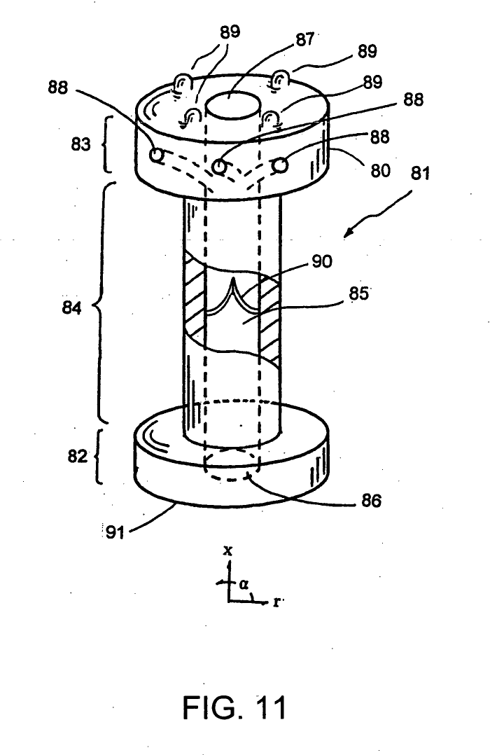

The trabecular stenting device 31 (or 81 as shown in FIG. 11) may be made by molding, thermo-forming, or other micro-machining techniques. The trabecular stenting device preferably comprises a biocompatible material. Biocompatible materials which may be used for the device preferably include, but are not limited to, titanium, medical grade silicone, e.g., SILASTIC™, available from Dow Coming Corporation of Midland, Mich.; and polyurethane, e.g., PELLETHANE™, also available from Dow Corning Corporation. In other embodiments, the device may comprise other types of biocompatible material, such as, by way of example, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like. In still other embodiments, composite biocompatible material may be used, wherein a surface material may be used in addition to one or more of the aforementioned materials. For example, such a surface material may include polytetrafluoroethylene (PTFE) (such as TEFLON™), polyimide, hydrogel, heparin, therapeutic drugs (such as beta-adrenergic antagonists and other anti-glaucoma drugs, or antibiotics), and the like.

As is well known in the art, a device coated or loaded with a slow-release (or time-release) substance can have prolonged effects on local tissue surrounding the device. The slow-release delivery can be designed such that an effective amount of substance is released over a desired duration. “Substance,” as used herein, is defined as any therapeutic or active drug that can stop, mitigate, slow-down or reverse undesired disease processes.

In one embodiment, the device may be made of a biodegradable (also including bioerodible) material admixed with a substance for substance slow-release into ocular tissues. In another embodiment, polymer films may function as substance containing release devices whereby the polymer films may be coupled or secured to the device. The polymer films may be designed to permit the controlled release of the substance at a chosen rate and for a selected duration, which may also be episodic or periodic. Such polymer films may be synthesized such that the substance is bound to the surface or resides within a pore in the film so that the substance is relatively protected from enzymatic attack. The polymer films may also be modified to alter their hydrophilicity, hydrophobicity and vulnerability to platelet adhesion and enzymatic attack.

Furthermore, the film may be coupled (locally or remotely) to a power source such that when substance delivery is desired, a brief pulse of current is provided to alter the potential on the film to cause the release of a particular amount of the substance for a chosen duration. Application of current causes release of a substance from the surface of the film or from an interior location in the film such as within a pore. The rate of substance delivery is altered depending on the degree of substance loading on the film, the voltage applied to the film, and by modifying the chemical synthesis of substance delivery polymer film.

The power-activated substance delivery polymer film may be designed to be activated by an electromagnetic field, such as, by way of example, NMR, MRI, or short range RF transmission (such as Bluetooth). In addition, ultrasound can be used to cause a release of a particular amount of substance for a chosen duration. This is particularly applicable to a substance coated device or a device made of a substrate containing the desired substance.

The device may be used for a direct release of pharmaceutical preparations into ocular tissues. As discussed above, the pharmaceuticals may be compounded within the device or form a coating on the device. Any known drug therapy for glaucoma may be utilized, including but not limited to, the following:
U.S. Pat. No. 6,274,138, issued Aug. 14, 2001, and U.S. Pat. No. 6,231,853, issued May 15, 2001, the entire contents of both of which are incorporated herein by reference, disclose the function of mitochondria and toxic substances synthesized as a metabolic byproduct within mitochondria of cells. Perry and associates (Perry H D et al. “Topical cyclosporin A in the management of postkeratoplasty glaucoma” Cornea 16:284-288, 1997) report that topical cyclosporin-A has been shown to reduce post-surgical increases in intraocular pressure. It is proposed that such compounds with known effects on mitochondrial stability might be effective in treating trabecular meshwork. An antagonistic drug to neutralize the toxic byproduct or a stabilizing drug to effect mitochondrial stability is believed able to restore the mitochondria function and subsequently mitigate the dysfunction of the trabecular meshwork.
U.S. Pat. No. 6,201,001, issued Mar. 13, 2001, the entire contents of which are incorporated herein by reference, discloses Imidazole antiproliferative agents useful for neovascular glaucoma;
U.S. Pat. No. 6,228,873, issued May 8, 2001, the entire contents of which are incorporated herein by reference, discloses a new class of compounds that inhibit function of sodium chloride transport in the thick ascending limb of the loop of Henle, wherein the preferred compounds useful are furosemide, piretanide, benzmetanide, bumetanide, torasernide and derivatives thereof;
U.S. Pat. No. 6,194,415, issued Feb. 27, 2001, the entire contents of which are incorporated herein by reference, discloses a method of using quinoxoalines (2-imidazolin-2-ylamino) in treating neural injuries (e.g. glaucomatous nerve damage);
U.S. Pat. No. 6,060,463, issued May 9, 2000, and U.S. Pat. No. 5,869,468, issued Feb. 9, 1999, the entire contents of which are incorporated herein by reference, disclose treatment of conditions of abnormally increased intraocular pressure by administration of phosphonylmethoxyalkyl nucleotide analogs and related nucleotide analogs;
U.S. Pat. No. 5,925,342, issued Jul. 20, 1999, the entire contents of which are incorporated herein by reference, discloses a method for reducing intraocular pressure by administration of potassium channel blockers;
U.S. Pat. No. 5,814,620, issued Sep. 29, 1998, the entire contents of which are incorporated herein by reference, discloses a method of reducing neovascularization and of treating various disorders associated with neovascularization. These methods include administering to a tissue or subject a synthetic oligonucleotide;
U.S. Pat. No. 5,767,079, issued Jun. 16, 1998, the entire contents of which are incorporated herein by reference, discloses a method for treatment of ophthalmic disorders by applying an effective amount of Transforming Growth Factor-Beta (TGF-beta) to the affected region;
U.S. Pat. No. 5,663,205, issued Sep. 2, 1997, the entire contents of which are incorporated herein by reference, discloses a pharmaceutical composition for use in glaucoma treatment which contains an active ingredient 5-[1-hydroxy-2-[2-(2-methoxyphenoxyl)ethylamino]ethyl]-2-methylbenzenesulfonamide. This agent is free from side effects, is stable, and has an excellent intraocular pressure reducing activity at its low concentrations, thus being useful as a pharmaceutical composition for use in glaucoma treatment;
U.S. Pat. No. 5,652,236, issued Jul. 29, 1997, the entire contents of which are incorporated herein by reference, discloses pharmaceutical compositions and a method for treating glaucoma and/or ocular hypertension in the mammalian eye by administering thereto a pharmaceutical composition which contains as the active ingredient one or more compounds having guanylate cyclase inhibition activity. Examples of guanylate cyclase inhibitors utilized in the pharmaceutical composition and method of treatment are methylene blue, butylated hydroxyanisole and N-methylhydroxylamine;
U.S. Pat. No. 5,547,993, issued Aug. 20, 1996, the entire contents of which are incorporated herein by reference, discloses that 2-(4methylaminobutoxy)diphenylmethane or a hydrate or pharmaceutically acceptable salt thereof have been found useful for treating glaucoma;
U.S. Pat. No. 5,502,052, issued Mar. 26, 1996, the entire contents of which are incorporated herein by reference, discloses use of a combination of apraclonidine and timolol to control intraocular pressure. The compositions contain a combination of an alpha-2 agonist (e.g., para-amino clonidine) and a beta blocker (e.g., betaxolol);
U.S. Pat. No. 6,184,250, issued Feb. 6, 2001, the entire contents of which are incorporated herein by reference, discloses use of cloprostenol and fluprostenol analogues to treat glaucoma and ocular hypertension. The method comprises topically administering to an affected eye a composition comprising a therapeutically effective amount of a combination of a first compound selected from the group consisting of beta-blockers, carbonic anhydrase inhibitors, adrenergic agonists, and cholinergic agonists; together with a second compound;
U.S. Pat. No. 6,159,458, issued Dec. 12, 2000, the entire contents of which are incorporated herein by reference, discloses an ophthalmic composition that provides sustained release of a water soluble medicament formed by comprising a crosslinked carboxy-containing polymer, a medicament, a sugar and water;
U.S. Pat. No. 6,110,912, issued Aug. 29, 2000, the entire contents of which are incorporated herein by reference, discloses methods for the treatment of glaucoma by administering an ophthalmic preparation comprising an effective amount of a non-corneotoxic serine-threonine kinase inhibitor, thereby enhancing aqueous outflow in the eye and treatment of the glaucoma. In some embodiments, the method of administration is topical, whereas it is intracameral in other embodiments. In still further embodiments, the method of administration is intracanalicular;
U.S. Pat. No. 6,177,427, issued Jan. 23, 2001, the entire contents of which are incorporated herein by reference, discloses compositions of non-steroidal glucocorticoid antagonists for treating glaucoma or ocular hypertension; and
U.S. Pat. No. 5,952,378, issued Sep. 14, 1999, the entire contents of which are incorporated herein by reference, discloses the use of prostaglandins for enhancing the delivery of drugs through the uveoscleral route to the optic nerve head for treatment of glaucoma or other diseases of the optic nerve as well as surrounding tissue. The method for enhancing the delivery to the optic nerve head comprises contacting a therapeutically effective amount of a composition containing one or more prostaglandins and one or more drug substances with the eye at certain intervals.

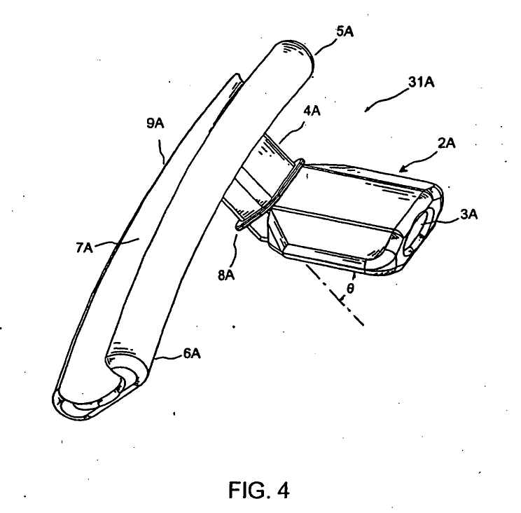

FIG. 4 illustrates another embodiment of a trabecular stenting device 31A which facilitates the outflow of aqueous from the anterior chamber 20 into Schlemm's canal 22, and subsequently into the aqueous collectors and the aqueous veins so that intraocular pressure is reduced. The device 31A comprises an inlet section 2A, a middle section 4A, and an outlet section 9A. The device 31A further comprises at least one lumen 3A traversing the sections 2A, 4A, 9A and providing fluid communication therebetween. The lumen 3A facilitates the transfer of aqueous from the inlet section 2A through the device 31A. The outlet section 9A is preferably curved, and may also be somewhat flexible, to facilitate positioning of the outlet section 9A within an existing outflow pathway of the eye 10. The outlet section 9A further comprises an elongate trough 7A for transmitting, or venting, aqueous. The elongate trough 7A is connected to and in fluid communication with the lumen 3A within the trabecular stenting device 31A.

A circumferential ridge 8A is provided at the junction of the inlet section 2A and the middle section 4A to facilitate stabilization of the device 31A once implanted within the eye 10. Preferably, the middle section 4A has a length (between the ridge 8A and the outlet section 9A) that is roughly equal to the thickness of the trabecular meshwork 21, which typically ranges between about 100 μm and about 300 μm, yet the length may be less than about 100 μm and greater than about 300 μm. In addition, the outlet section 9A may advantageously be formed with a protuberance or barb projecting therefrom so as to further stabilize the device 31A within the eye 10 without undue suturing.

As will be appreciated by those of ordinary skill in the art, the devices 31 and 31A may advantageously be practiced with a variety of sizes and shapes without departing from the scope of the invention. Depending upon the distance between the anterior chamber 20 and the drainage vessel (e.g., a vein) contemplated, the devices 31, 31A may have a length ranging from about 0.05 centimeters to over 10 centimeters, yet the length may be less than about 0.05 centimeters. Preferably, the devices 31 and 31A have an outside diameter ranging between about 30 μm and about 500 μm, with the lumens 7, 3A having diameters ranging between about 20 μm and about 250 μm, respectively. In some embodiments, the outside diameter may be less than about 30 μm and greater than about 500 μm, and the lumens 7, 3A may have diameters less than about 20 μm and greater than about 250 μm. In addition, the devices 31, 31A may have a plurality of lumens to facilitate transmission of multiple flows of aqueous. The inlet sections 2, 2A have longitudinal axes that form an angle (θ) ranging between about 20 degrees and about 150 degrees relative to the longitudinal axes of the middle sections 4, 4A, respectively. More preferably, the angles between the longitudinal axes of the inlet sections 2, 2A and the middle sections 4, 4A range between about 30 degrees and about 60 degrees, respectively. It is contemplated that the angle (θ) may be less than about 20 degrees and greater than about 150 degrees

One preferred method for increasing aqueous outflow in the eye 10 of a patient, to reduce intraocular pressure therein, comprises bypassing the trabecular meshwork 21. In operation, the middle section 4 of the device 31 is advantageously placed across the trabecular meshwork 21 through a slit or opening. This opening can be created by use of a laser, a knife, or other surgical cutting instrument. The opening may advantageously be substantially horizontal, i.e., extending longitudinally in the same direction as the circumference of the limbus 15 (FIG. 2). Other opening directions may also be used as well. The opening may advantageously be oriented at any angle, relative to the circumference of the limbus 15, that is appropriate for inserting the device 31 through the trabecular meshwork 21 and into Schlemm's canal 22 or other outflow pathway, as will be apparent to those skilled in the art. The middle section 4 may be semi-flexible and/or adjustable in position relative to the inlet section 2 and/or the outlet section 9, further adapting the device 31 for simple and safe glaucoma implantation. Furthermore, the outlet section 9 may be positioned into fluid collection channels of the natural outflow pathways. Such natural outflow pathways include Schlemm's canal 22, aqueous collector channels, aqueous veins, and episcleral veins. The outlet section 9 may be positioned into fluid collection channels up to at least the level of the aqueous veins, with the device inserted in a retrograde or antegrade fashion.

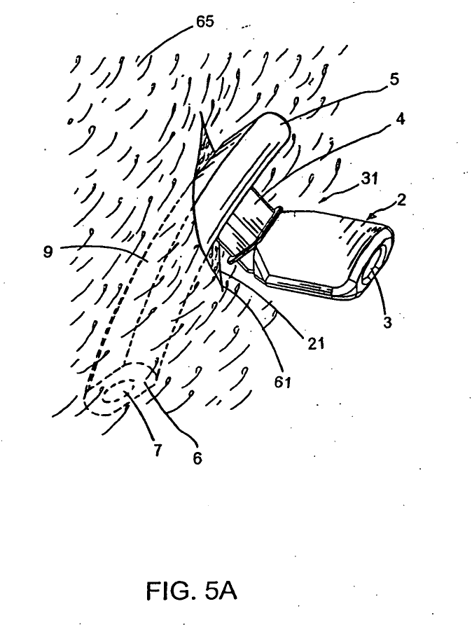

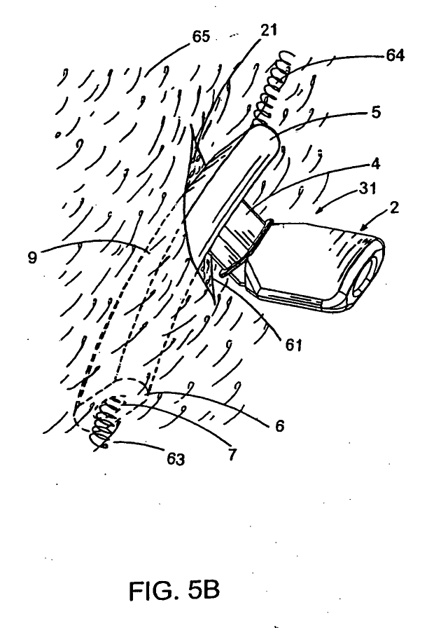

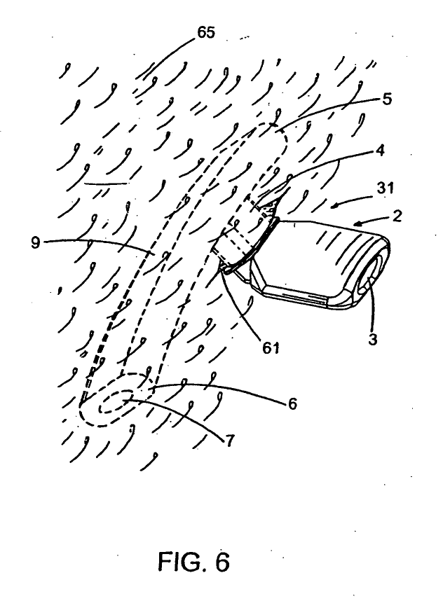

FIG. 5A generally illustrates a step in the implantation of the trabecular stenting device 31 through the trabecular meshwork 21. The outlet section 9 of the device 31 is inserted into an opening 61 in the trabecular meshwork 21. A practitioner may create the opening 61 “ab interno” from the interior surface 65 of the trabecular meshwork 21. The practitioner then advances the first outlet end 6 of the outlet section 9 through the opening 61 into a first side of Schlemm's canal 22 or other suitable outflow pathway within the eye 10. Next, the practitioner advances the second outlet end 5 through the opening 61 and into a second side of Schlemm's canal 22. The advancing of the second outlet end 5 may be facilitated by slightly pushing the second outlet end 5 through the opening 61. FIG. 6 generally illustrates a further stage in deployment of the device 31, wherein the entire outlet section 9 of the device 31 is implanted within Schlemm's canal 22, beneath the trabecular meshwork 21. At this stage, the lumen 3 of the implanted device 31 provides an enhanced fluid communication through the trabecular meshwork 21.

FIG. 5B shows an additional and/or alternate step in the implantation of the trabecular stenting device 31 through the trabecular meshwork 21. The practitioner inserts a distal end 63 of a guidewire 64 through the opening 61 into the first side Schlemm's canal 22. The practitioner then advances the first outlet end 6 of the outlet section 9 into Schlemm's canal 22 by “riding,” or advancing, the trabecular stenting device 31 on the guidewire 64. As will be apparent to those skilled in the art, the guidewire 64 will have a shape and size conforming to the shape and size of the lumen 7; and as such, may have an elliptical (e.g., oval) shape, a D-shape, a round shape, or an irregular (asymmetric) shape which is adapted for nonrotatory engagement for the device 31.

Another method for increasing aqueous outflow within the eye 10 of a patient, and thus reduce intraocular pressure therein, comprises: (a) creating an opening in the trabecular meshwork 21, wherein the trabecular meshwork 21 includes a deep side and superficial side; (b) inserting the trabecular stenting device 31 into the opening; and (c) transmitting aqueous through the device 31, to bypass the trabecular meshwork 21, from the deep side to the superficial side of the trabecular meshwork 21. This “transmitting” of aqueous is preferably passive, i.e., aqueous flows out of the anterior chamber 20 due to a pressure gradient between the anterior chamber 20 and the aqueous venous system 23.

Another method for increasing aqueous outflow within the eye 10 of a patient, and thus reduce intraocular pressure therein, comprises a) providing at least one pharmaceutical substance incorporated into a trabecular stenting device at about the middle section of the device; b) implanting the trabecular stenting device within a trabecular meshwork of an eye such that the middle section is configured substantially within the trabecular meshwork, the stenting device having a first end positioned in an anterior chamber of the eye while a second end is positioned inside a Schlemm's canal, wherein the first and the second ends of the trabecular stenting device establish a fluid communication between the anterior chamber and the Schlemm's canal; and c) allowing the middle section of the trabecular stenting device to release a quantity of said pharmaceutical substance into the trabecular meshwork.

It should be understood that the devices 31 and 31A are in no way limited to implantation within only Schlemm's canal 20, as depicted in FIGS. 5A and 5B. Rather, the devices 31 and 31A may advantageously be implanted within and/or used in conjunction with a variety of other natural outflow pathways, or biological tubular structures, as mentioned above. As will be apparent to those of ordinary skill in the art, the devices 31 and 31A may advantageously be used in conjunction with substantially any biological tubular structure without detracting from the scope of the invention.

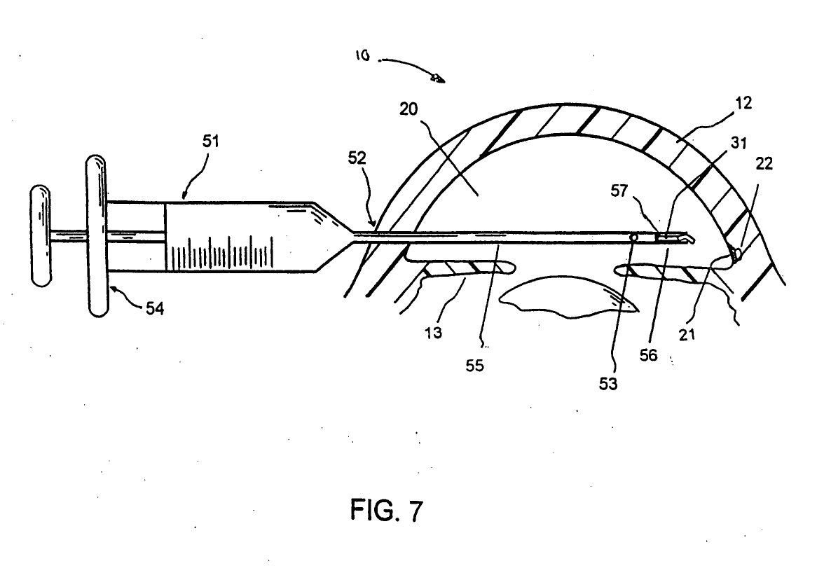

FIG. 7 generally illustrates a preferred method by which the trabecular stenting device 31 is implanted within the eye 10. In the illustrated method, a delivery applicator 51 is provided, which preferably comprises a syringe portion 54 and a cannula portion 55 which contains at least one lumen (not shown). The cannula portion 55 preferably has a size of about 30 gauge. However, in other embodiments, the cannula portion 55 may have a size ranging between about 16 gauge and about 40 gauge, and in yet further embodiments, the cannula portion 55 may have a size less than about 16 gauge and more than about 40 gauge. A distal section of the cannula portion 55 has at least one irrigating hole 53 in fluid communication with the lumen. A holder for holding the device 31 comprises a lumen 56 having a proximal end 57. In other embodiments, the holder may advantageously comprise a lumen, a sheath, a clamp, tongs, a space, and the like. The proximal end 57 of the lumen 56 is preferably sealed off from the remaining lumen and the irrigating hole 53 of the cannula portion 55. As will be recognized by those skilled in the art, however, in other embodiments of the cannula portion 55, the lumen 56 may advantageously be placed in fluid communication with the lumen and irrigating hole 53 of the cannula portion 55 without detracting from the invention.

In the method illustrated in FIG. 7, the device 31 is placed into the lumen 56 of the delivery applicator 51 and then advanced to a desired implantation site within the eye 10. The delivery applicator 51 holds the device 31 securely during delivery and releases it when the practitioner initiates deployment of the device 31.

In a preferred embodiment of trabecular meshwork surgery, a patient is placed in a supine position, prepped, draped, and appropriately anesthetized. A small incision 52 is then made through the cornea. The incision 52 preferably has a surface length less than about 1.0 millimeters in length and may advantageously be self-sealing. Through the incision 52, the trabecular meshwork 21 is accessed, wherein an incision is made with an irrigating knife (not shown). The device 31 is then advanced through the corneal incision 52 and across the anterior chamber 20, while the device 31 is held in the delivery applicator 51, under gonioscopic, microscopic, or endoscopic guidance. After the device 31 is appropriately implanted, the applicator 51 is withdrawn and the trabecular meshwork surgery is concluded.

FIG. 8 generally illustrates the use of the trabecular stenting device 31 for establishing an outflow pathway, passing from the anterior chamber 20 through the trabecular meshwork 21 to Schlemm's canal 22. As illustrated, an opening has been created in the trabecular meshwork 21. As will be appreciated by those of ordinary skill in the art, such an opening in the trabecular meshwork 21 may comprise an incision made with a microknife, a pointed guidewire, a sharpened applicator, a screw-shaped applicator, an irrigating applicator, a barbed applicator, and the like. Alternatively, the trabecular meshwork 21 may advantageously be dissected with an instrument similar to a retinal pick or microcurrette. Furthermore, the opening may advantageously be created by fiberoptic laser ablation. Referring again to FIG. 8, the outlet section 9 of the device 31 has been inserted in its entirety into the opening in the trabecular meshwork 21. The inlet section 2 is exposed to the anterior chamber 20, while the outlet section 9 is positioned near an interior surface 33 of Schlemm's canal 22. In other embodiments, the outlet section 9 may advantageously be placed into fluid communication with other natural outflow pathways, such as, but not limited to, aqueous collector channels, aqueous veins, and episcleral veins, as described above. A device such as the device 31A of FIG. 4, wherein the outflow section 9A has an open trough 7A for stenting purposes, may be used to maintain an opening of one or more of such natural outflows pathways. With the trabecular stenting device 31 implanted as illustrated in FIG. 8, aqueous flows from the anterior chamber 20 through the device 31 into Schlemm's canal 22, bypassing the trabecular meshwork 21, thereby reducing intraocular pressure within the eye 10.

Some aspects of the invention relate to a method of vasodilating a tissue of the aqueous cavity comprising administering a pharmaceutical agent to the aqueous cavity, wherein the vasodilating agent is a phosphodiesterase type 5 inhibitor or sertraline. In one embodiment, the phosphodiesterase type 5 inhibitor or sertraline is coated on a surface of a trabecular stent having a distal end and a proximal end, wherein the distal end of the trabecular stent is placed in an aqueous cavity and the proximal end of the trabecular stent is placed in an anterior chamber. In another embodiment, the aqueous cavity is selected from a group consisting of Schlemm's canal, a collector channel, and an aqueous vein. In still another embodiment, the trabecular stent is implanted ab internally.

FIG. 11 illustrates a preferred embodiment of a hollow trabecular microstent 81, which facilitates the outflow of aqueous from the anterior chamber 20 into Schlemm's canal 22, and subsequently into the aqueous collectors and the aqueous veins so that intraocular pressure is reduced. In the illustrated embodiment, the trabecular microstent 81 comprises an inlet section 82, having an inlet opening 86, an optional middle section 84, and an outlet section 83 having at least one opening 87, 88. The middle section 84 may be an extension of, or may be coextensive with, the inlet section 82. The device 81 comprises at least one lumen 85 within section 84, which is in fluid communication with the inlet opening 86 and the outlet opening 87, 88, thereby facilitating transfer of aqueous through the device 81. In one aspect, the outlet side openings 88, each of which is in fluid communication with the lumen 85 for transmission of aqueous, are arranged spaced apart around the circumferential periphery 80 of the outlet section 83. In another aspect, the outlet openings 88 are located and configured to enable jet-like infusing fluid impinging any specific region of Schlemm's canal tissue suitably for tissue stimulation.

As will be apparent to a person skilled in the art, the lumen 85 and the remaining body of the outlet section 83 may have a cross-sectional shape that is oval, circular, or other appropriate shape. Preferably, the middle section 84 has a length that is roughly equal to a thickness of the trabecular meshwork 21, which typically ranges between about 100 μm and about 300 μm, yet the length may be less than about 100 μm and greater than about 300 μm.

To further stent or open Schlemm's canal after implanting the axisymmetric device 81, a plurality of elevated (that is, protruding axially) supports or pillars 89 is located at the distal-most end of the outlet section 83 sized and configured for allowing media (for example, aqueous, liquid, balanced salt solution, viscoelastic fluid, therapeutic agents, or the like) to be transported freely.

The microstent 81 may further comprise a flow-restricting member 90 or an IOP pump (not shown), which is tightly retained within a lumen 85. The flow-restricting member 90 serves to selectively restrict at least one component in blood from moving retrograde, i.e., from the outlet section 83 into the anterior chamber 20 of the eye 10. Alternatively, the flow-restricting member 90 may be situated in any location within the device 81 such that blood flow is restricted from retrograde motion. The flow-restricting member 90 is sized and configured for maintaining the pressure of the infused fluid within the aqueous cavity for a suitable period of time. The flow-restricting member 90 may, in other embodiments, be a filter made of a material selected from the following filter materials: expanded polytetrafluoroethylene, cellulose, ceramic, glass, Nylon, plastic, and fluorinated material such as polyvinylidene fluoride (“PVDF”) (e.g., KYNAR™, by DuPont).

The trabecular microstent 31, 81 may be made by molding, thermo-forming, or other micro-machining techniques. The trabecular microstent preferably comprises a biocompatible material such that inflammation arising due to irritation between the outer surface of the device and the surrounding tissue is minimized. Biocompatible materials which may be used for the device preferably include, but are not limited to, titanium, stainless steel, medical grade silicone, e.g., SILASTIC™, available from Dow Coming Corporation of Midland, Mich.; and polyurethane, e.g., PELLETHANE™, also available from Dow Corning Corporation, as discussed with previous embodiments. In other embodiments, the device may comprise other types of biocompatible material, such as, by way of example, heparin, polyvinyl alcohol, polyvinyl pyrolidone, collagen, heparinized collagen, polytetrafluoroethylene, expanded polytetrafluoroethylene, fluorinated polymer, fluorinated elastomer, flexible fused silica, polyolefin, polyester, polysilicon, and/or a mixture of the aforementioned biocompatible materials, and the like. In another aspect, the microstent is made of a biodegradable material selected from a group consisting of poly(lactic acid), polyethylene-vinyl acetate, poly(lactic-co-glycolic acid), poly(D,L-lactide), poly(D,L-lactide-co-trimethylene carbonate), poly(caprolactone), poly(glycolic acid), and copolymer thereof.

In still other embodiments, composite biocompatible material may be used, wherein a surface material may be used in addition to one or more of the aforementioned materials. For example, such a surface material may include polytetrafluoroethylene (PTFE) (such as TEFLON™), polyimide, hydrogel, heparin, therapeutic drugs (such as beta-adrenergic antagonists, TGF-beta, and other anti-glaucoma drugs, or antibiotics), and the like.

As is well known in the art, a device coated or loaded with a slow-release substance can have prolonged effects on local tissue surrounding the device. The slow-release delivery can be designed such that an effective amount of substance is released over a desired duration.

In one embodiment, the device of the present invention may be made of a biodegradable (also including bioerodible) material admixed with a substance for substance slow-release into ocular tissues. In another embodiment, polymer films may function as substance containing release devices whereby the polymer films may be coupled or secured to the device. The polymer films may be designed to permit the controlled release of the substance at a chosen rate and for a selected duration, which may also be episodic or periodic. Such polymer films may be synthesized such that the substance is bound to the surface or resides within a pore in the film so that the substance is relatively protected from enzymatic attack. The polymer films may also be modified to alter their hydrophilicity, hydrophobicity and vulnerability to platelet adhesion and enzymatic attack.

The device may be used for a direct release of pharmaceutical preparations into ocular tissues. As discussed above, the pharmaceuticals may be compounded within the device or form a coating on the device. Any known drug therapy for glaucoma may be utilized.

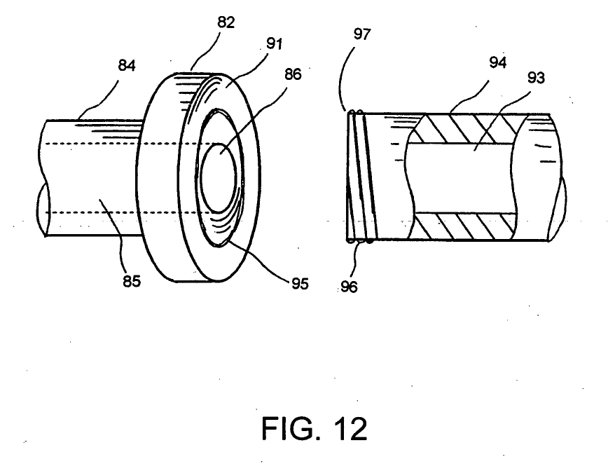

FIG. 12 shows a detailed view of the proximal section 82 of the microstent 81 of FIG. 11. In some aspect, the proximal section 82 has a bottom peripheral surface 91 that is about perpendicular to the lumen 85 of the microstent 81. A receiving thread arrangement 95 is appropriately located on the peripheral surface 91. The receiving thread arrangement 95 is sized and configured to releasably receive a screw thread arrangement 96 for coupling together, wherein the screw thread arrangement 96 is disposed at the distal end 97 of a fluid delivery element 94 which has a lumen 93 for transporting the infusing fluid into the aqueous cavity for therapeutic purposes. The coupling of the receiving thread arrangement 95 and the screw thread arrangement 96 makes the fluid infusion through the lumen 85 leak-proof enabling pressurization of the aqueous cavity.

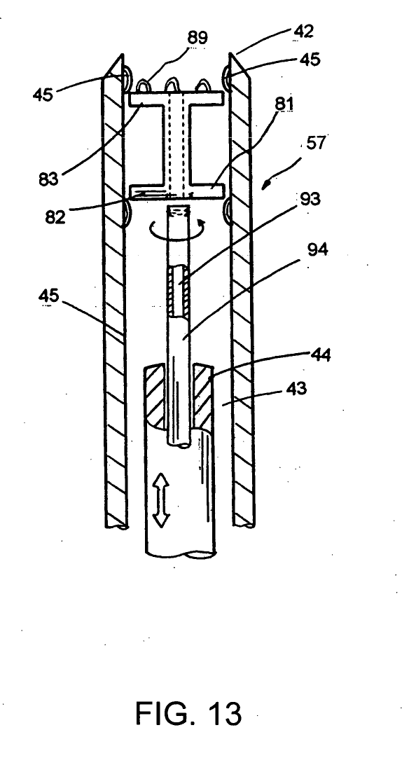

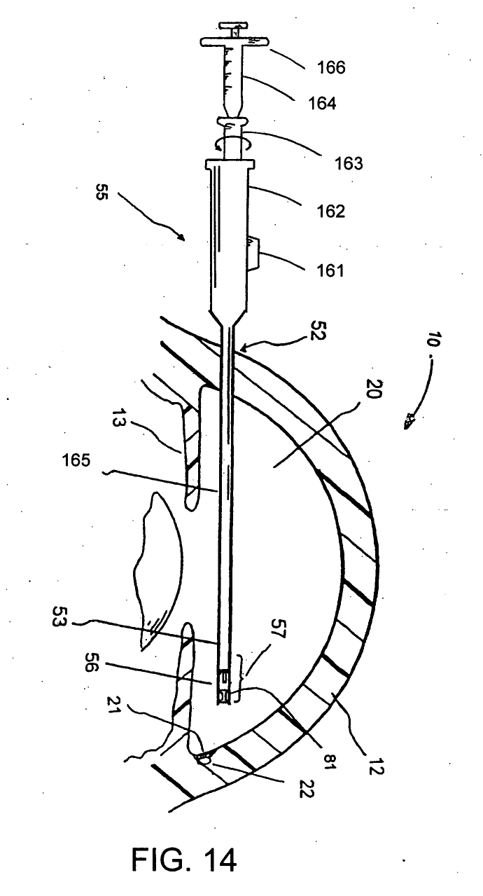

FIG. 13 shows a distal portion 57 of an applicator 55 for delivering a microstent 81 and infusing fluid for therapeutic treatment. The distal portion 57 comprises a distal cutting means 42 sharp enough for creating an incision on the cornea and also creating an opening on trabecular meshwork 21 for stent placement. The axisymmetric microstent 81 is snugly placed within the lumen 43 of the applicator 55 and retained by a plurality of stent retaining members 45. The microstent 81 is deployed from the applicator 55 once the distal section 83 passes beyond the edge of the trabecular meshwork 21. In one aspect, the stent deployment is facilitated by a plunger-type deployment mechanism 44 with an associated deployment actuator 161 mounted on the handle 162 of the applicator 55 (see FIG. 14).

The microstent 81 may be releasably coupled with a fluid delivery element 94 at any convenient time during the procedures. In one aspect, the screw-unscrew coupling steps between the microstent 81 and the fluid delivery element 94 is carried out by suitably rotating the fluid delivery element 94 with reference to the stent receiving thread arrangement 95, wherein the associated rotating mechanism 163 is located at the handle 162 of the applicator 55.

As will be appreciated by those of ordinary skill in the art, the device 81 may advantageously be practiced with a variety of sizes and shapes without departing from the scope of the invention. Depending upon the distance between the anterior chamber 20 and the drainage vessel (e.g., a vein) contemplated, the devices 81 may have a length ranging from about 0.05 centimeters to over 1 centimeter, yet the length may be less than about 0.05 centimeters. Preferably, the device 81 has an outside diameter ranging between about 30 μm and about 500 μm, with the lumen 85 having diameters ranging between about 20 μm and about 250 μm, respectively. In some embodiments, the outside diameter may be less than about 30 μm and greater than about 500 μm, and the lumen 85 may have a diameters less than about 20 μm and greater than about 250 μm. In addition, the device 81 may have a plurality of lumens to facilitate transmission of multiple flows of aqueous or infusing fluid.

One preferred method for increasing aqueous outflow in the eye 10 of a patient, to reduce intraocular pressure therein, comprises bypassing the trabecular meshwork 21. In operation, the middle section 84 of the device 81 is advantageously placed across the trabecular meshwork 21 through a slit or opening. This opening can be created by use of a laser, a knife, thermal energy (radiofrequency, ultrasound, microwave), cryogenic energy, or other surgical cutting instrument. The opening may advantageously be substantially horizontal, i.e., extending longitudinally in the same direction as the circumference of the limbus 15 (FIG. 2). Other opening directions may also be used, as well. The opening may advantageously be oriented at any angle, relative to the circumference of the limbus 15, that is appropriate for inserting the device 81 through the trabecular meshwork 21 and into Schlemm's canal 22 or other outflow pathway, as will be apparent to those skilled in the art. Furthermore, the outlet section 83 may be positioned into fluid collection channels of the natural outflow pathways. Such natural outflow pathways include Schlemm's canal 22, aqueous collector channels, aqueous veins, and episcleral veins.

FIG. 14 generally illustrates a preferred method by which the trabecular microstent 81 is implanted within the eye 10. In the illustrated method, a delivery applicator 55 is provided, which preferably comprises a syringe portion 164 and a cannula portion 165, which contains at least one lumen 43 in fluid communication with the fluid supply 166. The cannula portion 165 preferably has a size of about 30 gauge. However, in other embodiments, the cannula portion 165 may have a size ranging between about 16 gauges and about 40 gauges, and in yet other embodiments, the size may be less than about 16 gauges and greater than about 40 gauges. A holder 56 at the distal portion 57 of the cannula portion 165 for holding the device 81 may advantageously comprise a lumen, a sheath, a clamp, tongs, a space, and the like.

In the method illustrated in FIG. 14, the device 81 is placed into the lumen 43 of the delivery applicator 55 and then advanced to a desired implantation site within the eye 10. The delivery applicator 55 holds the device 81 securely during delivery and releases it when the practitioner initiates deployment actuator 161 of the applicator 55.

In a preferred embodiment of trabecular meshwork surgery, a patient is placed in a supine position, prepped, draped, and appropriately anesthetized. A small incision 52 is then made through the cornea 12 with a self-trephining applicator 55. The incision 52 preferably has a surface length less than about 1.0 millimeter in length and may advantageously be self-sealing. Through the incision 52, the trabecular meshwork 21 is accessed, wherein an incision is made with a cutting means 42 enabling forming a hole on the trabecular meshwork 21 for stent placement. The hole on the trabecular meshwork can also be created with a tip having thermal energy or cryogenic energy. After the device 81 is appropriately implanted, the applicator 55 is withdrawn and the trabecular meshwork surgery is concluded.

In some aspects, a method may be used for expanding or attenuating the capacity of the existing canal outflow system (also known as the “aqueous cavity”). This system could have become constricted or blocked due to age or other factors associated with glaucoma. In one aspect, a tight fluid coupling is established between an external pressured fluid source 166 and Schlemm's canal 22 through a microstent 81. It is also advantageous to connect the external pressurized fluid source through a removable instrument (for example, a temporary applicator, catheter, cannula, or tubing) to Schlemm's canal ab interno for applying the fluid infusion therapy.

Once the fluid coupling is established, the pressure in the canal, or other aqueous cavity, is raised by injecting fluid or fluid with therapeutic substances, such as vasodilation-enhancing agents. In some aspect of the present invention, a method is provided of treating glaucoma including infusing fluid into aqueous cavity from an anterior chamber end of a stent, wherein the fluid is at an elevated pressure above a baseline pressure of the aqueous cavity. The method further comprises placing a hollow trabecular microstent bypassing the trabecular meshwork, wherein the fluid is infused from the anterior chamber through a lumen of the microstent. The mode of fluid injection is selected from a group consisting of a pulsed mode, an intermittent mode, a programmed mode, or combination thereof. In one aspect, the pressure of the fluid therapy is effective to cause therapeutic effects on the tissue of the aqueous cavity. In another aspect, the fluid pressure is effective to cause the dilation of the aqueous cavity beyond the tissue elastic yield point for plastic permanent deformation. In other embodiment, the fluid is at an elevated pressure effective to cause plastic deformation for at least a portion of the aqueous cavity.

The fluid may be a salt solution such as Balanced Salt Solution, a viscoelastic (such as HEALON®), any other suitable viscous or non-viscous liquid, or suitable liquid loaded with drug at a concentration suitable for therapeutic purposes without causing safety concerns. A combination of liquids may also be used. The pressure is raised at an appropriate rate of rise to an appropriate level and for an appropriate length of time, as determined through development studies, to provide for the expansion of the outflow structures and/or a clearing of any blockages within them. The procedure can be augmented with other aids to enhance its effectiveness. These aids may include heat, vibration (sonic or ultrasonic), pulsation of a pressure front, pH, drugs, etc. It is intended that the aqueous cavity be expanded (attenuation or tissue stimulation) by this procedure resulting in an increased capacity for inflow and outflow of Schlemm's canal.

In some aspects, a method may implement a removable applicator, catheter, cannula, or tubing that is placed ab interno through the trabecular meshwork into the aqueous cavity of an eye adapted for infusing therapeutic liquid into the aqueous cavity.

In some aspects, a method of treating glaucoma may comprise providing at least one pharmaceutical substance incorporated into an axisymmetric trabecular microstent, implanting the microstent within a trabecular meshwork of an eye such that a first end of the microstent is positioned in an anterior chamber of the eye while a second end is positioned in a Schlemm's canal, wherein the first and second ends of the microstent establish a fluid communication between the anterior chamber and the Schlemm's canal, and allowing the microstent to release a quantity of the pharmaceutical substance into the eye. In one embodiment, the method further comprises a step of infusing fluid into the Schlemm's canal from the anterior chamber through a lumen of the microstent, wherein the fluid is at an elevated pressure above a baseline pressure of Schlemm's canal.

The Aqueous Veins

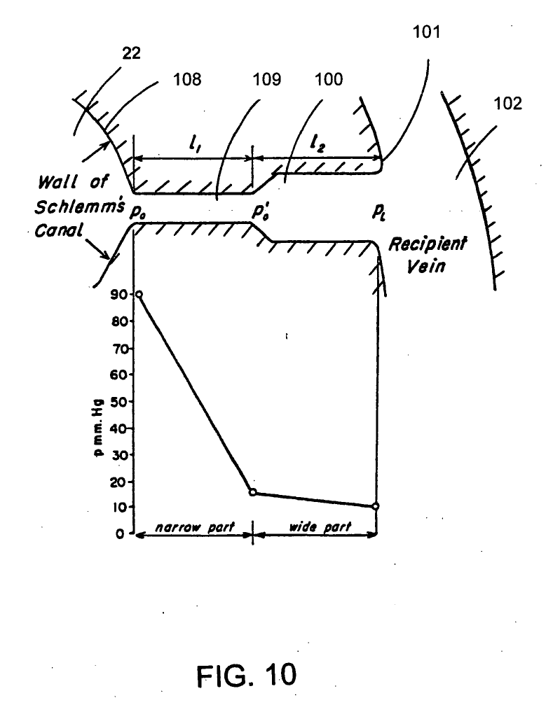

Schlemm's canal, outlet of the canal, and any downstream aqueous drainage passageway (that is, aqueous veins) are collectively called “aqueous cavity” in this invention. Aqueous veins are recognized by their pale or even colorless contents which contrast with the red color of ordinary blood vessels. The outlet and effect of outlet constriction is shown in FIG. 10 (from The Aqueous Veins: Biomicroscopic study of the aqueous humor elimination, a book by K W Ascher, published by Charles C Thomas, Springfield, Ill. 1961).

FIG. 10 shows effects of constriction of an outlet (109, 100) of Schlemm's canal 22. In one embodiment as shown, if the radius of the narrow tube 109 is 0.0065 mm and that of the wide tube is 0.013 mm and the length of both tubes equals 0.1 mm, the pressure drop along the constriction amounts to 94% of the total pressure drop along this tube. The nature of the junction of the outlet tube 109 with the wall 108 of Schlemm's canal 22 and the outlet tube 100 with the inner wall 101 of the recipient vein 102 will influence the flow of aqueous humor. The sharpness or roundness of the corners at the junctions and the angle of contact between the outlet tube and the vein into which aqueous empties will be factors of some importance. Any smooth muscle relaxation of the outlet tube 109, 100 along with the walls 108, 101 will significantly reduce the pressure drop over the outlet tube leading to enhanced aqueous outflow transportation due to increased pressure differential between the IOP and the extraocular aqueous pressure.

It has been shown that some anti-glaucomatous drugs increase the aqueous outflow and subsequently lower the IOP. Pilocarpine and eserine increase the output of clear fluid after administration into a glaucomatous eye. Some aspects relate to a method for enhancing pressure differential between an intraocular pressure and an extraocular aqueous pressure comprising lowering the extraocular aqueous pressure measured at certain points along the aqueous veins by relaxing the smooth muscle of the aqueous vein. In one embodiment, the step of relaxing the smooth muscle of the aqueous cavity is accomplished by slowly releasing smooth muscle relaxing drug at an effective dose over time.

The Smooth Muscle Relaxing Drugs

Features of the vasodilating substances may include the following. Adrenaline causes vasoconstriction mainly but vasodilation in skeletal muscle and coronary arterioles, and noradrenaline causes general vasoconstriction. Angiotensin II released from the kidney when blood volume or pressure falls causes general vasoconstriction. Antidiuretic hormone released by the anterior pituitary gland causes vasoconstriction of arterioles. Histamine, bradykinin and some prostaglandins released during the inflammatory process cause powerful vasodilation. Nitric oxide (NO), previously known as endothelium-derived relaxing factor (EDRF), is released by the endothelial lining of the blood vessels and acts locally as a vasodilator. Nitric oxide also regulates blood flow into the tissues. In cardiovascular disease, particularly arteriosclerosis, the endothelium is damaged and so nitric oxide release is impaired. Renin released from the kidneys causes vasoconstriction and promotes the retention of salt and water by the kidneys. This results in hypertension.

Vasodilation is the increase in the internal diameter of a blood vessel that results from relaxation of smooth muscle within the wall of the muscle. This causes an increase in blood flow but a decrease in vascular resistance. Vasodilation of the aqueous veins is effected via administering vasodilator to an eye by (1) a topically administered vasodilator agent onto an eye, (2) a slow-released (or time-released) vasodilator agent from an implant inside an eye, (3) retrograde infusion through aqueous veins, or (4) direct fluid infusion through a coupled microstent that bypasses the anterior chamber and/or trabecular meshwork.

Drugs for Ophthalmology Therapy-Sertraline

The antidepressant effect of sertraline is presumed to be linked to its ability to inhibit the neuronal reuptake of serotonin. It has only very weak effects on norepinephrine and dopamine neuronal reuptake. At clinical doses, sertraline blocks the uptake of serotonin into human platelets. Serotonin is a neurotransmitter. Sertraline HCl is a selective serotonin reuptake inhibitor (SSRI) for oral administration. It is chemically unrelated to other SSRIs, tricyclic, tetracyclic, or other available antidepressant agents. It has a molecular weight of 342.7. Sertraline hydrochloride has the following chemical name: (1S-cis)-4-(3,4-dichlorophenyl)-1,2,3,4-tetrahydro-N-methyl-1-nanphthalenamine hydrochloride. The empirical formula is C₁₇H₁₇NCl₂.HCl. Sertraline hydrochloride in oral administration is a white crystalline powder that is slightly soluble in water and isopropyl alcohol, and sparingly soluble in ethanol.

Serotonin (5-hydroxytryptamine, 5HT) is formed by the hydroxylation and decarboxylation of tryptophan. The greatest concentration of 5HT (90%) is found in the enterochromaffin cells of the gastrointestinal tract. Most of the remainder of the body's 5HT is found in platelets and the CNS. The effects of 5HT are felt most prominently in the cardiovascular system, with additional effects in the respiratory system and the intestines. Vasoconstriction is a classic response to the administration of 5HT. Some aspects of the invention provide sertraline as a vasoconstrictor antagonist through its uptake inhibition of serotonin, wherein the sertraline is topically administered onto an eye or slowly released through a sertraline-loaded implant within an eye.

Like most clinically effective antidepressants, sertraline downregulates brain norepinephrine and serotonin receptors in animals. In receptor binding studies, sertraline has no significant affinity for adrenergic (alpha(1), alpha(2) and beta), cholinergic, GABA, dopaminergic, histaminergic, serotonergic (5-HT1A, 5-HT1B, 5-HT2) or benzodiazepine binding sites. In placebo-controlled studies in normal volunteers, sertraline did not cause sedation and did not interfere with psychomotor performance.

The following are pharmacokinetics of the sertraline in oral administration. Following multiple oral once-daily doses of 200 mg, the mean peak plasma concentration (C(max)) of sertraline is 0.19 mcg/mL occurring between 6 to 8 hours post-dose. The area under the plasma concentration time is 2.8 mg hr/L. For desmethylsertraline, C(max) is 0.14 mcg/mL, the half-life 65 hours and the area under the curve 2.3 mg hr/L. Following single or multiple oral once-daily doses of 50 to 400 mg/day the average terminal elimination half-life is approximately 26 hours. Linear dose proportionality has been demonstrated over the clinical dose range of 50 to 200 mg/day. Food appears to increase the bioavailability by about 40%: it is recommended that sertraline be administered with meals.

Sertraline is extensively metabolized to N-desmethylsertraline, which shows negligible pharmacological activity. Both sertraline and N-desmethylsertraline undergo oxidative deamination and subsequent reduction, hydroxylation and glucuronide conjugation. Biliary excretion of metabolites is significant. Approximately 98% of sertraline is plasma protein bound. The interactions between sertraline and other highly protein bound drugs have not been fully evaluated. The pharmacokinetics of sertraline itself appear to be similar in young and elderly subjects. Plasma levels of N-desmethylsertraline show a 3-fold elevation in the elderly following multiple dosing, however, the clinical significance of this observation is not known.

Some aspects of the invention relate to the method of protecting optical retina nerves by slowly releasing loaded sertraline at an effective dose over time to the posterior segment site, e.g., by a retinal implant. Some aspects of the invention relate to a method of vasodilating a tissue of the aqueous cavity by administering a vasodilating agent to the aqueous cavity, wherein the vasodilating agent is sertraline.

Drugs for Ophthalmology Therapy-Pinacidil, Prazosin, Captopril

Several drugs were studied to show chronic responses of systemic hemodynamics and blood pressure counterregulatory mechanisms in matched groups of patients with essential hypertension (Am J. Cardiol 1987;60(4):303-308). Three drugs show equivalent decreases in mean arterial pressure compared with placebo baseline; they are pinacidil (a potassium channel opener as direct vasodilation), prazosin (or phenoxybenzamine, as alpha-adrenergic blockade) and captopril (as angiotensin-converting enzyme inhibition).

Some aspects of the invention relate to a method of dilating or relaxing the smooth muscle of the aqueous cavity accomplished by slowly releasing smooth muscle relaxing drug (for example, pinacidil, prazosin, captopril, and the like) at an effective dose over time to provide means for enhancing pressure differential between an intraocular pressure and an extraocular aqueous pressure (which results in enhanced aqueous outflow and lowed IOP). The smooth muscle relaxing agent may be topically administered onto an eye or slowly released from a drug-loaded implant within an eye.

Drugs for Ophthalmology Therapy-Sildenafil

Viagra, an oral therapy for erectile dysfunction, is the citrate salt of sildenafil, a selective inhibitor of cyclic guanosine monophosphate (cGMP)-specific phosphodiesterase type 5 (PDE5). Sildenafil citrate is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate. Sildenafil is also known as 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one. Sildenafil citrate is a white to off-white crystalline powder with a solubility of 3.5 mg/ml in water and a molecular weight of 666.7. Viagra is formulated as blue, film-coated, rounded, diamond-shaped tablets equivalent to 25 mg, 50 mg and 100 mg of sildenafil for conventional oral administration. In addition to the active ingredient, sildenafil citrate, each tablet contains the following inactive ingredients: microcrystalline cellulose, anhydrous dibasic calcium phosphate, croscarmellose sodium, magnesium stearate, hydroxypropyl methylcellulose, titanium dioxide, lactose, triacetin, and FD&C blue #2 aluminum lake.

Viagra promotes erections by relaxing the smooth muscle of the blood vessels thus increasing blood flow in the penis in response to sexual stimulation. It does this by specifically blocking a particular enzyme (protein that assists chemical reactions) called phosphodiesterase type 5 (PDE 5). This is the enzyme that normally breaks down chemicals causing the erectile response. Therefore, by blocking the breakdown of erectile chemicals, the drug promotes a harder and more prolonged erection. The effects of sildenafil to cause relaxation of smooth muscle of the aqueous veins provide means for enhancing pressure differential between an intraocular pressure and an extraocular aqueous pressure. Some aspects of the invention relate to the method of dilating or relaxing the smooth muscle of the aqueous cavity accomplished by slowly releasing smooth muscle relaxing drug (for example, sildenafil citrate or its analog) at an effective dose over time, either topical administration onto the eye, drug infusion through a lumen of an implant by a syringe, or through controlled release from an implant.

Recently, researchers learned that a drug widely used for impotence, sildenafil, may also be effective as a selective vasodilator of the pulmonary system. Sildenafil is an inhibitor of PDE 5, a hormone produced by the body that causes blood vessels to constrict. Researchers believe that because of the high concentrations of PDE 5 in the lungs, sildenafil may function in a similar manner to nitric oxide. Nitric oxide (NO), previously known as endothelium-derived relaxing factor (EDRF), is released by the endothelial lining of the blood vessels in association with sildenafil and acts locally as a vasodilator.

Drugs for Ophthalmology Therapy-Vardenafil

Vardenafil, an oral therapy for erectile dysfunction, is a selective inhibitor of cyclic guanosine monophosphate (cGMP)-specific phosphodiesterase type 5 (PDE5). Vardenafil is designated chemically as 2-[2-ethoxy-5-(4-ethyl-piperazin-1-yl-1-sulphonyl)-phenyl]-5-methyl-7-propyl-3H-imidazo[5,1-f][1,2,4]triazin-4-one. It is also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, (i.e., the compound of examples 20, 19, 337 and 336 of published international application WO 99/24433; the compound of example 11 of published international application WO 93/07124). Some aspects of the invention relate to the method of administrating vardenafil by slowly releasing the drug at an effective dose over time, either topical administration onto the eye, drug infusion through a lumen of an implant by a syringe, or through controlled release from an implant.

Drugs for Ophthalmology Therapy-Tadalafil

Tadalafil, an oral therapy for erectile dysfunction, is a selective inhibitor of cyclic guanosine monophosphate (cGMP)-specific phosphodiesterase type 5 (PDE5). Tadalafil is designated chemically as (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione, (i.e. the compound of examples 78 and 95 of published international application WO95/19978).

Some aspects of the invention relate to a method of vasodilating a tissue of the aqueous cavity comprising administering a vasodilating agent to the aqueous cavity. In one aspect, the vasodilating agent is a phosphodiesterase type 5 inhibitor selected from a group consisting of 5-(2-ethoxy-5-morpholinoacetylphenyl)-1-methyl-3-n-propyl-1,6-dihydro-7-H-pyrazolo[4,3-d]pyrimidin-7-one; 3-ethyl-5-[5-(4-ethylpiperazin-1-ylsulphonyl)-2-n-propoxyphenyl]-2-(pyridin-2-yl)methyl-2,6-dihydro-7H-pyrazolo[4,-3-d]pyrimidin-7-one; 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one; (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazol-o[4,3-d]pyrimidin-7-one, also known as 3-ethyl-5-{5-[4-ethylpiperazin-1-yl-sulphonyl]-2-([(1R)-2-methoxy-1-methylethyl]oxy)pyridin-3-yl}-2-methyl-2,-6-dihydro-7H-pyrazolo[4,3-d]pyrimidin-7-one; 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridyl-sulphonyl}-4-ethylpiperazine; 5-[2-iso-Butoxy-5-(4-ethylpiperazin-1-yl-sulphonyl)pyridin-3-yl]-3-ethyl-2-(1-methylpiperidin-4-yl)-2,6-dihydro-7H-p-yrazolo[4,3-d]pyrimidin-7-one; 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one; 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one; 5-(5-Acetyl-2-butoxy-3-pyridinyl)-3-ethyl-2-(1-ethyl-3-azetidinyl)-2,6-di-hydro-7H-pyrazolo[4,3-d]pyrimidin-7-one. Further, a phosphodiesterase type 5 inhibitor may be selected from a group consisting of sildenafil, vardenafil and tadalafil.

Compound Composition

If a combination of active agents are administered, then they may be administered simultaneously, separately, or sequentially. The compounds of the invention can be administered alone but, in human therapy will generally be administered in admixture with a suitable pharmaceutical excipient diluent or carrier selected with regard to the intended route of administration and standard pharmaceutical practice. The sildenafil, pinacidil, prazosin, captopril, sertraline, vardenafil, and/or tadalafil (“compounds”) can be incorporated into various types of ophthalmic formulations for topical delivery to the eye. They may be combined with ophthalmologically acceptable preservatives, surfactants, viscosity enhancers, penetration enhancers, buffers, sodium chloride, and water to form aqueous, sterile ophthalmic suspensions or solutions. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a thickener, such as, hydroxymethylcellulose, hydroxyethylcellulose, hydroxypropylmethylcellulose, methylcellulose, polyvinyl-pyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated. Preparation of such topical formulations are well described in the art of pharmaceutical formulations as exemplified, for example, by Remington's Pharmaceutical Science, Edition 17, Mack Publishing Company, Easton, Pa.

If dosed topically, the compounds are preferably formulated as topical ophthalmic suspensions or solutions, with a pH of about 4 to 8, preferably about 7. The compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the routine discretion of a skilled clinician.

The preferred compound, sildenafil, pinacidil, prazosin, captopril or sertraline, may be mixed with an IOP-lowering agent for treating glaucoma patients. The IOP-lowering agents useful in the present invention include all presently known IOP-lowering pharmaceuticals, including, but not limited to, miotics (e.g., pilocarpine, carbachol, and acetylcholinesterase inhibitors); alpha and alpha/beta adrenergic agonists (e.g., epinephrine, dipivalylepinephrine, para-amino clonidine and brimonidine); beta-blockers (e.g., betaxolol, S-betaxolol, levobunolol, carteolol, and timolol); prostaglandins and their analogues and derivatives, such as, compounds disclosed in U.S. Pat. No. 4,599,353; No. 5,093,329; and No. 5,321,128; and carbonic anhydrase inhibitors (e.g., acetazolamide, methazolamide, and ethoxzolamide, and compounds disclosed in U.S. Pat. No. 5,153,192; U.S. Pat. No. 5,240,923; U.S. Pat. No. 5,378,703; and U.S. Pat. No. 4,797,413) and ocular hypertensive lipids, such as those compounds (neutral replacement of the carboxylic acid group of prostaglandin F2α e.g. AGN 192024) described in IOVS, Mar. 15, 1998, Vol. 39, No. 4; WO 97/30710, U.S. Pat. Nos. 5,238,961; 5,262,437; 5,328,933; 5,352,708; 5,312,842; 5,552,434; 5,545,665; 5,688,819. The preferred IOP-lowering agents are: timolol, betaxolol, S-betaxolol levobunolol, carteolol, pilocarpine, carbachol, epinephrine, dipivalyl epinephrine-α methyl dipivalylepinephrine, brinzolamide, dorzolamide, unoprostone, latanoprost, travoprost, apraclonidine, and brimonidine.

The compound (sildenafil, pinacidil, prazosin, captopril or sertraline) with one or more IOP-lowering agents will be administered topically at a concentration of between 0.001 and 5.0 wt %, preferably, 0.01 to 2.5 wt %, but preferably 0.001-0.005 for prostaglandins.

In addition to the compound (sildenafil, pinacidil, prazosin, captopril or sertraline), the additional active ingredient(s) that can be included in the compositions of the present invention include all ophthalmic, dermatological, otic or nasal agents that can be topically applied, retrogradely infused or coated onto a trabecular stent. For example, such ophthalmic agents include (but are not limited to): anti-glaucoma agents, such as beta-blockers (e.g., betaxolol, S-betaxolol, levobunolol, carteolol, timolol and combination thereof), prostaglandins (e.g., metabolite derivatives of arachindonic acid), miotics (e.g., pilocarpine, carbachol, acetylcholinesterase inhibitors and combination thereof), sympathomimetics (e.g., epinephrine and dipivalylepinephxine), carbonic anhydrase inhibitors (e.g., acetazolamide, methazolamide, ethoxzolamide and combination thereof), carbonic anhydrase inhibitors (e.g., acetazolamide, methazolamide, ethoxzolamide and combination thereof), dopaminergic agonists and antagonists, and alpha and alpha/beta adrenergic receptor agonists (e.g., epinephrine, dipivalylepinephrine, para-amino clonidine, brimonidine and combination thereof); anti-infectives, such as ciprofloxacin; non-steroidal and steroidal anti-inflammatories, such as suprofen, ketorolac, dexamethasone, rimexolone and tetrahydrocortisol; proteins; growth factors, such as EGF; and anti-allergic agents, such as cromolyn sodium, emedastine and olopatadine. Compositions of the present invention may also include combinations of active ingredients.

The compositions of the present invention can also include other components, for example, pharmaceutically acceptable buffers; tonicity agents; comfort-enhancing agents; solubilizing aids; pH adjusting agents; antioxidants; and stabilizing agents. The compositions may also contain additional preservatives (in conjunction with the cationic preservatives addressed above). As will be appreciated by those skilled in the art, the compositions may be formulated in various dosage forms suitable for topical delivery, including solutions, suspensions, emulsions, and gels. Some aspects of the invention relate to a method of vasodilating a tissue of an aqueous cavity comprising administering a vasodilating agent to the aqueous cavity, wherein the vasodilating agent is administered topically on an eye configured for diffusing to the aqueous cavity. Although some of the drugs disclosed herein may not effectively drop intraocular pressure at physiological doses, super-physiological (pharmacological) doses can be administered in an attempt to titrate to an effective dose.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

1. A method of lowering intraocular pressure, the method comprising:
positioning an end of a body in an aqueous cavity of an eye; and
introducing a dilating agent from the body into the aqueous cavity of the eye;
wherein the dilating agent is selected from the group consisting of a phosphodiesterase inhibitor, an alpha adrenergic antagonist, a serotonin reuptake inhibitor, and an angiotensin converting enzyme inhibitor.

2. The method of claim 1, wherein the dilating agent is introduced through a lumen in the body.

3. The method of claim 1, wherein the dilating agent is time-released from the body.

4. The method of claim 1, wherein the aqueous cavity comprises the trabecular meshwork

5. The method of claim 1, wherein the aqueous cavity comprises Schlemm's canal.

6. The method of claim 1, wherein the aqueous cavity comprises an aqueous collector channel.

7. The method of claim 1, wherein the aqueous cavity comprises an episcleral vein.

8. The method of claim 1, further comprising introducing a fluid through a lumen in the body into Sclemm's canal of the eye.

9. The method of claim 8, further comprising performing a viscocanalostomy through the lumen of the body.

10. The method of claim 1, further comprising introducing the body into the anterior chamber of the eye through a corneal incision prior to positioning the body in the aqueous cavity.

11. The method of claim 1, wherein the dilating agent comprises sildenafil.

12. The method of claim 1, wherein the dilating agent comprises vardenafil.

13. The method of claim 1, wherein the dilating agent comprises tadalafil.
